(19) United States
(12) Patent Application Publication
(10) Pub. No.: US 2012/0177686 A1
(43) Pub. Date: Jul. 12, 2012

(54) VACCINE FOR THE PREVENTION OF ACUTE LYMPHOBLASTIC LEUKEMIA

(76) Inventor: Detlef Oerter, Berlin (DE)

(21) Appl. No.: 13/386,159

(22) PCT Filed: Jul. 20, 2010
(86) PCT No.: PCT/EP2010/060472

§ 371 Date: Mar. 27, 2012

(30) Foreign Application Priority Data
Jul. 23, 2009 (DE) 10 2009 034 553.1

(51) Int. Cl. A61K 39/125; A61P 31/14; A61P 35/02; A61P 37/04

(52) U.S. Cl. 424/216.1

(57) ABSTRACT

The present invention relates to a coxsackie vaccine or a vaccine based on coxsackie B viruses for the prevention of acute lymphoblastic leukemia (ALL), which occurs in particular in children in the age from the second birthday to the fifth birthday.

The present invention relates to a vaccine based on coxsackie B viruses for the prevention of acute lymphoblastic leukemia (ALL). This disease occurs in particular in children in the age group from the second to the fifth year of life.

The coxsackie viruses belong to the genus of the enteroviruses, to which the polioviruses and the echoviruses also belong. Coxsackie viruses can be divided into two subgroups: coxsackie viruses of type A with 23 serotypes and coxsackie viruses of type B with 6 serotypes.

Coxsackie viruses was isolated at the end of the 1940s for the first time from the stool of children from the US-American town of Coxsackie, who show signs of paralysis. Based on different effects on newborn mice, the isolated viruses were divided into the two aforementioned groups A and B. Whereas coxsackie viruses of group A generally cause inflammatory reactions, coxsackie viruses of type B infect a large number of tissues and organs in humans and animals and can lead to a rapid killing of the tissue. On infection of newborn mice in animal tests they lead after some days to a paralysis and death.

The transmission of viruses of the genus Enterovirus is mainly feco-oral or e.g. in the form of droplet infections. It is known that coxsackie viruses in humans generally cause infectious diseases with a harmless course, such as ordinary cold, but also other diseases such as hand-foot-and-mouth disease. Coxsackie B viruses can, however, also cause meningitis, pancreatitis or myocarditis, more rarely also paralyses. Coxsackie viruses of type B are distributed world-wide and are known causative agents of a number of diseases in humans. Coxsackie B viruses are discussed in the literature also in connection with viral-induced heart muscle inflammations (myocarditis) and viral-induced diabetes mellitus type I (IDDM).

Thus, coxsackie B4 viruses are suspected of being the cause of a viral myocarditis, which can sometimes also prove fatal.

The RNA genome and the structure of the capsule protein of various serotypes of coxsackie viruses have already been identified and described. Experimental vaccines for coxsackie B viruses also already exist, e.g. also based on cDNA-immunizations, but at present still no permitted vaccine is obtainable on the market.

Acute lymphoblastic leukemia (ALL) is a rare disease, but it represents a life-threatening cancer disease of childhood. Based on specific markers, four types of ALL can be distinguished. ALL occurs in particular in the industrialized countries and up to 80 to 90% of cases in children in the age group from the second to the fifth year of life (Rossig et al. Radiation Protection Dosimetry, 2008, 132, 114-118). This distribution is also designated as “childhood peak”. In the developing countries, ALL occurs far more rarely, and the “childhood peak” described is not formed here.

Approximately 800 children fall ill with acute lymphoblastic leukemia of childhood (ALL) in Germany annually, out of more than 500 000 births (Kaatsch et al., Radiation Protection Dosimetry, 2008, 132, 107-113) and in the USA approx. 3000 children, wherein most fall ill between the second and fifth year of life. By cytostatic and radiotherapy treatment, on average 80% of children with ALL are primarily cured. These exhausting and lengthy conventional cancer therapies have, along with the relatively high failure rates, considerable side effects, for example in the form of disturbances of thyroid function and impairment of neurocognitive capacities. Moreover, some of the originally cured patients later suffer a secondary tumor.

For the reasons presented above, there is an urgent need for new methods and medicaments for the prevention of this disease, which would moreover mean a great medical advance. As the causes that lead to an ALL disease (etiology of ALL) are still unknown, despite intensive research, to date there are no vaccines or medicinal products that combat the disease causally.

An aim of the present invention is to provide a safe and well tolerated vaccine for the prevention of ALL in children. It has now been found that coxsackie B viruses represent a causative agent for acute lymphoblastic leukemia (ALL) in children and that by a targeted immunization (vaccination) against coxsackie B viruses, falling ill with acute lymphoblastic leukemia (ALL) can be prevented.

The invention relates moreover to the use of a vaccine based on coxsackie B viruses for the prevention of acute lymphoblastic leukemia (ALL).

It was found that the characteristics of acute lymphoblastic leukemia (ALL) coincide with the features that have been described for infections and diseases due to coxsackie B viruses. Moreover, a two-stage course of infection by coxsackie B viruses in connection with ALL diseases became clear. Infection twice, first in the womb, preferably in the embryonic phase from the 3rd to the 8th week of pregnancy and a second infection after the first year of life with the identical serotype, is, as is presented in the following, often responsible for the development of ALL.

If children who have already come into contact in the embryonic phase with coxsackie B viruses, come into contact again, for example in the first year of life, with coxsackie viruses, no coxsackie-induced disease, such as ALL, occurs. There is a sufficient, passive immunization protection by the antibodies transferred from the mother. If, however, a child who had contact in the embryonic stage with a coxsackie B virus, comes into contact after the first year of life with the identical coxsackie B virus, with which its mother had contact, it has a far higher chance of falling ill with ALL.

About 5 to 10% of pregnant women in the industrialized countries experience a clinically unremarkable infection with coxsackie B viruses. As the coxsackie B virus can cross the placental barrier, infection of the embryo can also occur. This leads, owing to the lymphocytotropism of the coxsackie B virus, to an increased occurrence of chromosomal abnormalities in the precursors of the mature lymphocytes in the embryo.

According to observation, the proportion of those children that come into contact with coxsackie B virus in the first year of life do not fall ill, as the mother's antibodies transferred passively at birth represent a protection against diseases by coxsackie B virus. The children infected in the first year of life form additional new antibodies, which represent a long lasting protection.

The proportion of the children that comes into contact, after the first year of life, with the coxsackie B virus of the same serotype with which their mother had contact, falls ill more intensively with ALL. As molecular-genetic investigations elucidate, about 1% of the children who suffer a chromosomal abnormality of the lymphocytes in utero, show a transition to a malignant degeneration of the lymphocytes (ALL disease), i.e. 1% of about 5%, which is equivalent to about 0.05% (Mori et al., Proc Natl Acad Sci USA, 2002, 99, 8242-8247).

It can be shown that the characteristics of ALL can be linked causally with the characteristics of a two-stage infection with coxsackie B viruses described above. In particular the following characteristics of ALL can be correlated with an infection with coxsackie B viruses, wherein a causal contribution of coxsackie B viruses can be demonstrated:

General Rarity of ALL:

As described above, ALL is a rare disease, which results from the special two-stage course of ALL caused by coxsackie B viruses described above. A small percentage of 5-10% of pregnant women in the industrialized countries experience a generally clinically unremarkable infection with coxsackie B viruses. The children who suffered, in the embryonic stage, an infection with coxsackie B viruses and thus a chromosomal abnormality of the lymphocytes, and do not come into contact with coxsackie B virus of the identical serotype until after the first year of life, can fall ill with ALL.

Particular rarity of ALL in developing countries with absence of “childhood peak”:

ALL occurs far more rarely in developing countries than in the industrialized countries, and in the developing countries the so-called “childhood peak” is absent.

In the developing countries, owing to inadequate hygiene, in particular the lack of flushing toilets and disposable diapers, most children have an infection with coxsackie B viruses in the first year of life. Owing to the passive protection by maternal antibodies described above and the renewed immunization, ALL diseases do not occur.

Occurrence of the “Childhood Peak” after the First World War in the Industrialized Countries:

The “childhood peak” described above occurred in England and in the white population in the USA after the first world war. In the Afro-American population group in the USA and in Japan, the “childhood peak” was not observed until after the second world war.

This temporal occurrence coincides with the distribution of flushing toilets and the increasing hygiene in the stated countries/population groups. This increasing hygiene prevents contact with coxsackie B viruses in the first year of life.

The research by Smith (Smith et al., Cancer causes and Control, 1998, 9, 285-298) show the temporal variations in the hepatitis A virus (HAV) infection rates, wherein the HAV infection pattern serves as indicator for the fecal-oral transmission route, in relation to the variations in mortality and incidence of childhood leukemia in various countries. The investigation comes to the conclusion that improved conditions of public hygiene go together with higher rates of childhood leukemia. As coxsackie B viruses are mainly transmitted by fecal-oral routes, this work supports the two-stage course of infection with coxsackie B viruses described in the present application and the causal relationship of ALL and infections with coxsackie B viruses.

In a Japanese study (Watanabe et al., Kansenshogaku Zasshi, 1982, 56, 977-981) it is for example presented that in the period 1960-1962 still 50% of children under one year had contact with coxsackie B viruses. In contrast, in the period 1977-1980, none of the children under one year had contact with coxsackie B viruses.

ALL as Monoclonal Disease of the Lymphocytes:

ALL affects almost exclusively the precursors of the lymphocytes, so a causative agent must have high affinity to lymphocytes. The coxsackie B virus has a definite lymphocytotropism.

Decrease of ALL Risk Through Early Kindergarten Attendance:

Review works come to the conclusion that there is a link between kindergarten attendance and contracting ALL, and that in particular an early start of kindergarten attendance, i.e. before the 3rd month of life and between the 3rd and 6th month of life, reduces the risk of ALL disease more than late kindergarten attendance (Ma et al., British journal of cancer, 2002, 86, 1419-1424; Urayama et al., Int J Epidemiol, 2010, 39, 718-32). Attending a community establishment in the first year increases the probability of contact and a clinically unnoticed infection with coxsackie B viruses. According to the two-stage route of infection described above, this represents a protection against ALL disease.

Seasonal Influence on ALL:

The authors of the review work (McNally et al., Brit J Haematol, 2004, 127, 243-263) show a link between the month of birth and the occurrence of ALL diseases. Out of four studies of the seasonality of the month of birth, three show a peak in February to April. Only one gives a peak in late summer.

In another study, all cases of ALL in the north of England between 1968 and 2005 are analyzed with respect to seasonality of birth. Here, a statistically significant increase is shown for the month of birth March for 1 to 6 year old children with ALL (Basta et al., Paediatr Perinat Epidemiol, 2010, 24, 309-18).

As in England and Denmark the coxsackie B viruses show a clear disease peak in the third quarter of the calendar year, the first four to eight embryonic weeks would correspond to a sensitive phase with respect to increased ALL risk. This coincides with the time point of the particular frequency of occurrence of the precursors of lymphopoiesis in the embryo.

Moreover, the results of studies that describe the seasonality of ALL diagnosis are in good agreement with the temporal peak of coxsackie B virus infections in the third quarter of the year.

Space-Time Clustering of ALL Diseases:

Some investigations concern the question of a significant increase in ALL diseases with respect to space and time. In many cases the investigations point to a significant so-called space-time clustering of ALL diseases. A review work of McNally (McNally et al., Int. cancer, 2009, 124, 449-455) comes to the conclusion that the results of space-time clustering and of spatial clustering of ALL diseases are consistent for a number of ALL infections in particular for the “childhood peak”. Space-time clustering is also described for disease cases through coxsackie B viruses.

Influence of Previous Spontaneous Abortions on ALL Disease:

Various works (van Steensel-Moll et al., Int J Epidemiol, 1985, 14, 555-559; Kaye et al., Cancer, 1991, 68, 1351-1355; Yeazel et al., Cancer, 1995, 75, 1718-1727) investigate whether a spontaneous abortion has an influence on whether the subsequently born child falls ill with ALL. The works come to the conclusion that a spontaneous abortion increases the risk that the next-born child falls ill with ALL. The link of spontaneous abortions and recent infections with coxsackie B viruses is shown in a Swedish study, but there is no information on infection with other viruses (Frisk, G., Diderholm, H., J Infect, 1992, 24, 141-145; Axelsson et al., J Med Virology, 1993, 39, 282-285).

Influence of Socio-Economic Living Standard and Disease with ALL:

In the review work of McNally (McNally et al. (Brit J Haematology, 2004, 127, 243-263), a positive association is found between acute leukemia and higher socio-economic living standard.

The two-stage infection process described above and the fact that the probability of contact with coxsackie B viruses in the first year of life is slight in children with high socio-economic living standard, explain a slightly higher rate of ALL diseases in children with high socio-economic living standard.

Clinical Unremarkableness of the Causative Agent for ALL Diseases:

As to date no link of ALL with a viral disease has been recognized, the virus causing ALL must cause, both in the mother and in the child after the first year of life (which then falls ill with ALL later), clinically either no symptoms at all, or must develop under a nonspecific clinical picture, such as an ordinary cold (“common cold”) or acute gastroenteritis. The majority of coxsackie B infections are either clinically silent or proceed under the clinical picture of an ordinary cold (“common cold”) or acute gastroenteritis.

In a scientific study of Greaves and Buffler (Greaves, M., Buffler, P., A., Brit J Cancer, 2009, 100, 863), it is stressed that in contrast to the results of Cardwell (Cardwell et al., Brit J Cancer, 2008, 99, 1529-1533); which made no reference to the “delayed infection” hypothesis of Greaves, the protection by infections in the first year of life can certainly be caused by clinically silent, i.e. asymptomatic infections. The protection against ALL by an infection with coxsackie B viruses in the first year of life, described in the present application, is in good compliance with Greaves' assumption, as an infection with coxsackie B viruses proceeds asymptomatically in the great majority of cases (Danes et al., J Hyg Epidemiol Microbiol Immun, 1983, 27, 163-172). According to a hypothesis proven by cytogenetic investigations and generally accepted (“double hit” theory, Greaves loc. cit.), at least two events lead to ALL: an infection during pregnancy (“first hit”), and a subsequent second postnatal infection (“second hit”). The two-step infection with a coxsackie B virus presented above is in very good agreement with this “double hit” theory.

Increase in Malformations in Children with ALL:

The work of Miller (Miller R., W., New Engl J Med, 1963, 268, 393-401) refers to a small, but significant increase in malformations in children with ALL.

It is known that coxsackie B viruses, which as described cross the placental barrier and can infect the embryo, can in rare cases cause malformations in humans.

Influence of other Viral Diseases in the First Year of Life:

It was shown that roseola infections, which are also known as three-day fever (roseola infantum), and often occur in infancy or very early childhood, and ear infections, such as otitis media, in the first year of life, lower the risk of falling ill with ALL.

It is known that, inter alia, coxsackie B viruses can also cause a middle-ear inflammation (otitis media). Furthermore, it is described that roseola infections (roseola infantum, (normally triggered by the human herpes virus) are a consequence of a coxsackie infection. According to the two-stage course of infection with coxsackie B viruses described above, contact with coxsackie B viruses in the first year of life represents a protection against ALL disease.

ALL epidemic at the Beginning of the 1970s:

From the end of the 1960s to the beginning of the 1970s, there is in the industrialized countries an increase in cases of ALL, which disappears again at about the middle of the 1970s. Between 1963 and 1969 there is a pronounced coxsackie B epidemic.

The increase in incidence of leukemia in the first year of life is slight between 1920 and 2000, but the increase in leukemia cases between the second and fifth birthday is considerable. The slight increase in cases of leukemia in the first year of life (“infant leukemia”) is probably only a reflection of improved access to diagnosis, whereas the large increase between the second and fifth birthday (“childhood peak”) is without any doubt based on a real increase. The de facto absent increase in cases of “infant leukemia” in contrast to the “childhood peak” supports very well the hypothesis presented above, that the two-step infection is an infection with the identical serotype: Infection during the first year of life with the identical coxsackie B virus therefore cannot lead to a disease, as the infant is protected against an infection with coxsackie B viruses by the passively transferred antibodies of the mother during the first year of life. The antibodies passively transferred at birth have disappeared from the child towards the end of the first year of life, the child is now susceptible to a renewed infection with the coxsackie B virus, with which it first had contact during embryogenesis. This explains why the “childhood peak” in all publications does not begin until after the first birthday.

The present invention relates to a vaccine based on coxsackie B viruses as medicinal product for the prevention of acute lymphoblastic leukemia (ALL), in particular of acute lymphoblastic leukemia (ALL) in children.

In particular the vaccine finds application in the prevention of acute lymphoblastic leukemia of children, which occurs in the age group from the second to the fifth year of life.

Vaccines based on coxsackie B viruses are to be understood, in the sense of the present application, as compositions that contain a coxsackie B virus-specific antigen produced biologically or by genetic engineering. These may be killed coxsackie B viruses, protein and/or nucleic acid fragments (such as cDNA or RNA) of coxsackie B viruses and coxsackie B virus-specific viral genomes, in which a nucleotide sequence coding for cytokines, such as interferon-gamma, has been inserted. In the sense of the present invention, so-called killed or live vaccines can be used.

In a preferred embodiment of the invention the vaccine is a composition based on coxsackie B viruses and is applied as killed vaccine (e.g. inactivated coxsackie B viruses) for the prevention of acute lymphoblastic leukemia (ALL) in children.

In particular, killed vaccines based on coxsackie B viruses are suitable for triggering a specific immune response, without causing a disease due to coxsackie B viruses.

Attenuation or reduction of virulence means the intentional decrease in virulence, i.e. the disease-causing properties of the pathogen, wherein its capacity for multiplying (live-attenuated) and its antigenic properties are largely preserved. A distinction is generally made between cold-adapted strains, which can only multiply at temperatures around 25° C., and temperature-sensitive strains, which can only multiply in a temperature range of about 38-39° C.

Killed vaccines contain as a rule inactivated or killed pathogens, which are not capable of multiplying. For example, the following types of killed vaccines can be used:

Inactivated Whole-Particle Vaccines (Whole Virus Vaccine):

The inactivation (killing) of the viruses takes place by means of chemical substances or combinations of substances, e.g. formaldehyde, beta-propiolactone, psoralen, wherein the virus envelope is retained.

(Inactivated) Partial-Particle Vaccines:

There takes place a cleavage of the virus envelope with detergents or organic solvents and optionally an inactivation with chemical substances.

Subunit Vaccines:

The surface of the viruses is completely dissolved and specific protein components isolated. Subunit vaccines are only slightly immunogenic, but have little side effects.

In addition to the possibility of using complete killed (inactivated) or weakened (attenuated) pathogens as antigens in a vaccine, there is also the possibility of triggering the desired immune response with nucleic acid or protein fragments of the pathogens. Thus, in recent years, methods for production of cDNA vaccines based on viral or bacterial cDNA have been described in the literature. An advantage of the so-called cDNA vaccination is the extensive avoidance of side effects of the usual methods of vaccination.

In the literature a distinction is generally made between an active immunization (active vaccination, active vaccine) and a passive immunization (passive vaccination, passive vaccine). In active vaccination, a weakened form of the disease or an immune response is achieved artificially by administering pathogens that are or are not capable of multiplying. In passive vaccination, immunoglobulin preparations or the serum of actively immunized humans or animals are administered (parenterally, intravenously), wherein specific antibodies for the treatment or prevention of infectious diseases are transferred.

The present invention relates to a vaccine based on coxsackie B viruses as vaccine or medicinal product for the prevention of acute lymphoblastic leukemia (ALL), in particular of acute lymphoblastic leukemia (ALL) in children, wherein an active and/or passive vaccine can be used.

The present invention relates in particular to a vaccine based on coxsackie B viruses and a medicinal product for the prevention of acute lymphoblastic leukemia (ALL), in particular of acute lymphoblastic leukemia (ALL) in children, wherein an active vaccine finds application. In particular the vaccine contains one or more antigens selected from killed coxsackie B viruses, protein and/or nucleic acid fragments (in particular cDNA fragments) of coxsackie B viruses and coxsackie B virus-specific viral genomes, in which a nucleotide sequence coding for cytokines, such as interferon-gamma, has been inserted.

The relationship between the occurrence of ALL and the two-stage infection process with coxsackie B viruses described above (first prenatally, then after the first year of life) can in particular be demonstrated using “Guthrie cards”. The Guthrie test is among the screening tests of the newborn used throughout the world, in which as a rule around the 3rd day of life of the child a heel blood sample is taken. With this blood, a filter paper card is impregnated in predetermined fields. This dry blood sample is then investigated with respect to various metabolic disorders (such as phenylketonuria). Often these filter paper cards are stored in the clinics for many years and can still be used for blood tests even years after the birth. Viruses or virus-specific antibodies can also be detected in these dried blood samples from the newborn, for example the cytomegalovirus. In Guthrie cards of ALL patients, increased indications are found of a coxsackie B virus infection of the mother during pregnancy.

The occurrence of ALL in children is definitely connected with the presence of coxsackie B viruses or coxsackie B virus-specific antibodies. Moreover, differentiation is possible with respect to the various serotypes of coxsackie B viruses.

In a preferred embodiment of the invention, the vaccine based on coxsackie B viruses (for the prevention of acute lymphoblastic leukemia) is a vaccine containing at least one coxsackie B virus-specific antigen selected from the group consisting of killed (inactivated) coxsackie B viruses, protein fragments of coxsackie B viruses and nucleic acid fragments (in particular cDNA fragments) of coxsackie B viruses.

In particular, the vaccine based on coxsackie B viruses can contain coxsackie B virus-specific antigens, in particular killed coxsackie B viruses, of at least one serotype selected from the group coxsackie B1 viruses, coxsackie B2 viruses, coxsackie B3 viruses, coxsackie B4 viruses, coxsackie B5 viruses and coxsackie B6 viruses.

An embodiment is further preferred in which the vaccine based on coxsackie B viruses contains specific antigens, in particular killed coxsackie B viruses, of at least one serotype selected from the group coxsackie B2 viruses, coxsackie B3 viruses, coxsackie B4 viruses and coxsackie B5 viruses.

It may be advantageous if the vaccine brings about an immunization against all six known serotypes of the coxsackie B virus. Preferred in the sense of the invention is therefore also a vaccine containing a combination of two, three, four or even more antigens, which is specific in each case for one of the coxsackie B virus serotypes B1, B2, B3, B4, B5 and B6. In this sense, a preferred embodiment of the invention is directed at a vaccine based on coxsackie B viruses, which contains coxsackie B virus-specific antigens, in particular killed coxsackie B viruses, of the serotypes coxsackie B1 viruses, coxsackie B2 viruses, coxsackie B3 viruses, coxsackie B4 viruses, coxsackie B5 viruses and coxsackie B6 viruses.

Preferably the vaccine described above based on coxsackie B viruses contains protein and/or nucleotide fragments of coxsackie B viruses of at least one serotype selected from the group coxsackie B2 viruses, coxsackie B3 viruses, coxsackie B4 viruses and coxsackie B5 viruses.

The vaccine described in the present application for the prevention of acute lymphoblastic leukemia in children is administered to the children preferably before completion of the first year of life, in particular before completion of the sixth month of life. Therefore the vaccine described for the prevention of acute lymphoblastic leukemia is suitable for administration in children before completion of the first year of life, in particular before completion of the sixth month of life.

The vaccination takes place preferably intramuscularly or subcutaneously in the 3rd, 4th and 5th month of life of the child, and in a booster vaccination in the 11th and 18th year of life of the child.

The vaccine based on coxsackie B viruses disclosed in the present description can be administered in an application form known for vaccines, in particular for active vaccines. In principle the vaccine according to the invention can be administered parenterally, wherein in particular consideration may be given to intramuscular and subcutaneous administration. An intramuscular application of the vaccine is preferred.

In a further preferred embodiment, the described vaccine based on coxsackie B viruses for the prevention of acute lymphoblastic leukemia is suitable for intramuscular administration in children before completion of the sixth month of life.

The present invention relates furthermore to the use of a vaccine based on coxsackie B viruses for the production of a composition for the prevention of acute lymphoblastic leukemia in children.

The methods for the production of a vaccine based on coxsackie B viruses are known in principle to a person skilled in the art. For the production of inactivated coxsackie B viruses, for example mammalian cell cultures (for example monkey kidney cells (e.g. Vero cells), human diploid lung cell lines (WI-238, MRCS) can be grown and can be inoculated with coxsackie viruses of a single (or several) serotypes. In this, it is necessary to pay attention to a strict standardization of the production conditions (composition of the culture media, cell density, age of the cultures at inoculation, amount of the virus inoculated per cell, incubation temperature and time, number of multiplication cycles per production pass etc.). In particular after 48 to 72 hours, the virus harvest takes place by removal of the supernatant liquid under sterile conditions. These can then be filtered e.g. through a small filter pore size (e.g. of 0.22 micrometer), in order to remove larger cell residues. Optionally the liquid volume can be adapted to the necessary virus concentration, the virus suspension frozen until use and/or stored at low temperatures (e.g. of about 4° C.).

Optionally the thus obtained viruses can be killed for instance by heat or chemicals, or be weakened by means of usual methods of virus attenuation. The inactivation of the coxsackie B viruses can for example take place by treatment with chemicals such as formaldehyde, by high-energy radiation or heat treatment.

Another production method of the vaccine is based on the insertion of the encoding sequences of cytokines (such as interferon-gamma) in the viral genome of the coxsackie B virus. A vaccination with a coxsackie B3 strain altered in this way proved very effective in the animal-experimental prevention of coxsackie B3-induced myocarditides.

For the final formulation, aliquots of the virus suspension can be mixed with a suitable stabilizer, in particular with sorbitol, to achieve the recommended dosage, wherein viral constituents should be present in a concentration that produce a sufficient immune response in the human body. The dosage of the vaccine is based on the application form and is in principle familiar to a person skilled in the art.

Additionally to the coxsackie B virus-specific antigen, which produces the desired immune response in the human body, the vaccines described in the present application contain pharmaceutically compatible carriers and excipients. Can be used for instance: phenoxyethanol, magnesium chloride, aluminum salts, carbohydrates such as sucrose, preservatives such as antibiotics, thiomersal, phenol, formaldehyde.

The application-ready formulated vaccine can for example be filled in ampules and be stored until use.

The present application further relates to a method of production of a vaccine based on coxsackie B viruses for the prevention of acute lymphoblastic leukemia (ALL) in children, wherein coxsackie B viruses selected from at least one serotype are mixed with a pharmaceutically compatible carrier.

The term “chromosomal preparation” as used in the present invention, relates to the preparation of chromosomes or aberrations thereof. Chromosomes can be prepared from all tissues capable of dividing, the so-called sample material, e.g. peripheral blood, tissue (fibroblasts), amniotic fluid, chorionic villi, bone marrow. Preferably, lymphocytes from peripheral blood are suitable for chromosomal preparation, as the taking of venous blood is simple and the mitosis yield in cultivation is very good. As the lymphocytes present in the blood do not normally divide, however, in the culture they must be stimulated to division by a mitogen (e.g. colchicine). The techniques of chromosomal preparation are variable and generally known, but are based basically on the following steps: optionally setting up a culture with the special culture medium and in a suitable culture vessel; optionally wait for the culture time, optionally change of the culture medium and checking of the number of mitoses; stopping of growth by means of colchicine (spindle poison); treatment of the vital cells with hypotonic solution; fixing with an acetic acid-methanol mixture; optionally applying the cells on an object slide; making visible by staining the preparations, or excitation of fluorescence in FISH. So as also to detect cryptogenic chromosomal aberrations, for example translocations from chromosome to chromosome (in the case of ALL translocation from chromosome 12 to 21 fluorescence-in-situ-hybridization is used.

The term “fluorescence-in-situ-hybridization” (FISH), as used in the present invention, relates to a molecular-biological method, for detecting nucleic acids, i.e. RNA or DNA, in tissues, individual cells or on metaphase chromosomes by means of fluorescence. In this, an artificially produced probe from a nucleic acid is used, which hybridizes, thus binds, via base pairings to the nucleic acid to be detected. The designation “in situ” is used, as the detection is carried out directly in the respective structure. Basically the number, position and activity of genes can be determined or also whole chromosomes can be marked by means of chromosomal-in-situ-suppression (CISS) hybridization, and whole genomic DNA can be used as probe, genomic-in-situ-hybridization (GISH). The underlying mechanism of FISH can be described as follows: DNA is chemically constructed as linear polyester from deoxyribose and phosphoric acid with heterocyclic nitrogen bases in the side chain, so-called single strand. Two such DNA-single strands can form a double helix by hydrogen-bridge bonds. Hydrogen bridges of the double helix can be separated into two single strands by heating to temperatures around approx. 80° C. or by addition of organic solvents such as formamide. The temperature at which this separation of the double strand occurs is designated as melting temperature. After a separation, two DNA single strands can recombine again specifically to the double strand, if the base sequence of the single strands is complementary to each other. This specific recombination of DNA single strands of different origin to the double strand is called hybridization.

A hybridization site can be detected by attaching a fluorescence dye to a DNA molecule and, after completed hybridization, exciting this to fluorescence with a suitable light source. If a biological preparation is used as target DNA, by this specific recombination it is possible to detect a completely determined DNA sequence e.g. in a chromosome or in a cell nucleus. This special hybridization variant is called fluorescence in situ hybridization (FISH).

The present invention is described in more detail by the following examples.

### EXAMPLE 1

### Production of a Vaccine Based on Inactivated Coxsackie B Viruses

The production process of an inactivated coxsackie B vaccine comprises the following steps:
production of mammalian cell cultures;
virus-seeding/virus-inoculation;
virus-growth (incubated at 37° C.);
virus-harvest;
clarification; concentration; purification by gel-permeation chromatography; and ion exchange chromatography;—
filtration;
addition of vaccines other serotypes;
final formulation.

The coxsackie B virus is obtained from the stool of a child recently fallen ill with ALL and for example multiplied in Vero cells (normal kidney cells of the African green monkey). After microcarrier cultures, the medium is removed, the cells are washed and infected with the seed (multiplicity of infection, MOI).

After inoculation, the host cells are incubated at 37° C. for 72 to 96 hours. For harvest, the virus liquid is removed from the microcarrier (e.g. by sedimentation or with a special filter). A coarse purification step follows, to remove the majority of the contaminating cells. Then fine purification by chromatography takes place. In the first coarse purification step, the virus liquid is clarified first, to remove the coarse cellular debris. For this, a series of different filters is used, wherein the last filter is a 0.2 μm filter. The thus pre-clarified virus suspension is then concentrated by ultrafiltration (cut off of 100 kD), to reduce the volume of the liquid.

In the chromatographic purification, different principles can be applied and combined; for example a separation based on the molecular weight of the material by gel filtration and a separation based on the ionic charge by ion exchange chromatography. The antigen content of the liquid is monitored. For this, the concentration of contaminating proteins is monitored after each step, in order to monitor the compliance of the purified product. As cell lines are used for the production of this vaccine, the elimination of nucleic acids is of particular importance. The DNA clearance factor after the last purification step must be 10⁸, which means that the end product contains less than 10 pg DNA per dose.

In the next step, the inactivation of the purified virus suspension takes place, wherein in the sense of quality and safety of the vaccine it must be ensured that no live viruses are present in the end product. Simultaneously, however, in the inactivation process, for example in a chemical inactivation process, the antigenicity of the virus particles must be maintained. The inactivation takes place at the end of the purification process, in order to prevent a crosslinking of contaminants with the virus particles. The chemical inactivation can for example take place with formaldehyde, which is put for instance for inactivation of other enteroviruses such as the poliomyelitis viruses.

Before the chemical inactivation, the purified virus suspension is sterilized by filtration. This filtration sterilization is not longer than 27 hours the amount of formaldehyde required for inactivation added under aseptic conditions to the purified and sterilized virus suspension. Then this mixture is incubated at 37° C. for 6 days. The suspension is then filtered a second time and incubated again (6 to 9 days). Inactivation temperature: 37° C., formaldehyde concentration in final dilution 1:4000, pH of the medium 7.0.

Before the final formulation of the vaccine, the inactivated virus suspension is stored at 4° C. During this time, samples are taken, to determine the complete virus inactivation. The production steps described above are carried out similarly for all six serotypes of the coxsackie B virus. After verification of the inactivation step, the monovalent coxsackie B vaccines of the serotypes are pooled.

### EXAMPLE 2

### Animal-Experimental Tests

Pregnant mice are inoculated intraperitoneally with a defined coxsackie B virus strain and half of their progeny are inoculated with the identical strain (first group), the other 50% with a coxsackie B virus strain that belongs to another serotype (second group). It can be shown that a lymphatic leukemia occurs in the first group, not in the second.

### EXAMPLE 3

### Viroserological Investigations

a) Virus Serology in Mothers

Blood samples are taken from mothers of ALL patients and control mothers and the frequencies of coxsackie B infections are determined comparatively. It can be shown that the proportion of mothers with infections with coxsackie B viruses in the mothers of ALL patients is higher than in the control group.

b) Virus Detection in Guthrie Cards

The frequencies of coxsackie B infections in ALL patients and in a control group are determined using Guthrie cards. It can be demonstrated that IgM antibodies, which indicate a recent infection with coxsackie B, can be found with higher frequency in Guthrie cards of ALL patients. This supports the role of the coxsackie B viruses in the “first hit”, i.e. in the first stage of the described two-stage course of infection.

c) Virus Serology for ALL Patients

The frequency of coxsackie B infections is determined, in particular by the detection of IgM antibodies, in ALL patients and in a control group. It can be demonstrated that IgM antibodies are to be found with higher frequency in ALL patients. This supports the role of the coxsackie B viruses in the “second hit”, i.e. in the second stage of the described two-stage course of infection.

d) Virus Serology in the Stool of ALL Patients

The frequency of coxsackie B viruses is determined in the stool of ALL patients and in a control group. It can be demonstrated that coxsackie B viruses are to be found with higher frequency in ALL patients. This supports the role of the coxsackie B viruses in the “second hit”, i.e. in the second stage of the described two-stage course of infection

e) Virus Detection in the Lymphocytes of ALL Patients

Detection of coxsackie B viruses or parts of these in the lymphocytes of ALL patients and of control children: In the lymphocytes of ALL patients, the detection for coxsackie B viruses can be provided with a higher frequency. This supports the role of the coxsackie B viruses in the “second hit”, i.e. in the second stage of the described two-stage course of infection

### EXAMPLE 4

### Biological Evidence

Biological evidence was provided by testing whether human lymphoblasts from umbilical cord blood react to an exposure with coxsackie B viruses in vitro with such chromosomal aberrations, as are found in routine diagnostics in patients with ALL of children, and whether an exposure with coxsackie A viruses does not lead to such chromosomal aberrations necessary for predisposition to ALL.

Samples of human umbilical cord blood of the company PromoCell, Heidelberg, were used. The samples were cultured in the incubator at 37 degrees Celsius and 5% CO₂, then inoculated with a defined amount of coxsackie B viruses, or coxsackie A viruses, and cultured further. Then it was topped up with PBS (phosphate buffered salt solution) and centrifuged for 10 minutes at 778×g. The buffy-coat was removed, the cells were washed and brought into the culture preparation with phythaemagglutinin.

A) The chromosomal preparation is based on the enrichment of metaphase cells by arresting the cells in mitosis by adding the spindle poison colchicine. By treatment of the still vital cells with hypotonic solution, a swelling of the cells caused by osmosis takes place, which are then fixed with a mixture of methanol and vinegar and applied on the object slide. Then the analysis of the chromosomes, or their aberrations, takes place.

Result for A: about 70% with ALL specific chromosomal aberrations present were found.

B) For detecting cryptogenic chromosomal aberrations, fluorescence-in-situ-hybridization (FISH) was necessary. This relates in particular to the commonest chromosomal aberration of ALL, namely translocation of chromosome 12 to 21.

Result: frequency: in 15%, translocation of chromosome 12 to 21 was found.

1. A vaccine based on coxsackie B viruses for the prevention of acute lymphoblastic leukemia (ALL).

2. The vaccine based on coxsackie B viruses as claimed in claim 1, characterized in that it is a question of the prevention of acute lymphoblastic leukemia of children, which occurs in children in the age group from the second to the fifth year of life.

3. The vaccine based on coxsackie B viruses as claimed in claim 1, characterized in that it is a vaccine containing at least one antigen selected from the group consisting of killed coxsackie B viruses, protein fragments of coxsackie B viruses and cDNA fragments of coxsackie B viruses.

4. The vaccine based on coxsackie B viruses as claimed in claim 1, characterized in that it is a vaccine containing killed coxsackie B viruses of at least one serotype selected from the group coxsackie B1 viruses, coxsackie B2 viruses, coxsackie B3 viruses, coxsackie B4 viruses, coxsackie B5 viruses and coxsackie B6 viruses.

5. The vaccine based on coxsackie B viruses as claimed in claim 1, characterized in that it is a vaccine containing killed coxsackie B viruses of at least one serotype selected from the group coxsackie B2 viruses, coxsackie B3 viruses, coxsackie B4 viruses and coxsackie B5 viruses.

6. The vaccine based on coxsackie B viruses as claimed in claim 1, characterized in that it is a vaccine containing weakened and/or killed coxsackie B viruses of the serotypes coxsackie B1 viruses, coxsackie B2 viruses, coxsackie B3 viruses, coxsackie B4 viruses, coxsackie B5 viruses and coxsackie B6 viruses.

7. The vaccine based on coxsackie B viruses as claimed in claim 1, characterized in that it is a vaccine containing protein and/or nucleotide fragments of at least one coxsackie B virus serotype selected from the group coxsackie B1 viruses, coxsackie B2 viruses, coxsackie B3 viruses, coxsackie B4 viruses, coxsackie B5 viruses and coxsackie B6 viruses.

8. The vaccine based on coxsackie B viruses as claimed in claim 1, characterized in that the vaccine for the prevention of acute lymphoblastic leukemia is suitable for administration in children before completion of the first year of life.

9. The vaccine based on coxsackie B viruses as claimed in claim 1, characterized in that the vaccine for the prevention of acute lymphoblastic leukemia is suitable for administration in children before completion of the sixth month of life.

10. The vaccine based on coxsackie B viruses as claimed in claim 1, wherein the vaccine for the prevention of acute lymphoblastic leukemia is suitable for intramuscular or subcutaneous administration in children before completion of the sixth month of life.

11. A method of production of a vaccine based on coxsackie B viruses for the prevention of acute lymphoblastic leukemia in children, wherein inactivated coxsackie B viruses selected from at least one serotype are mixed with a pharmaceutically compatible carrier.

12. A method for the prevention of acute lymphoblastic leukemia in children comprising the step of administering a vaccine based on coxsackie B viruses.

13. The vaccine based on coxsackie B viruses as claimed in claim 2, characterized in that it is a vaccine containing at least one antigen selected from the group consisting of killed coxsackie B viruses, protein fragments of coxsackie B viruses and cDNA fragments of coxsackie B viruses.

14. The vaccine based on coxsackie B viruses as claimed in claim 3, characterized in that it is a vaccine containing killed coxsackie B viruses of at least one serotype selected from the group coxsackie B1 viruses, coxsackie B2 viruses, coxsackie B3 viruses, coxsackie B4 viruses, coxsackie B5 viruses and coxsackie B6 viruses.

15. The vaccine based on coxsackie B viruses as claimed in claim 4, characterized in that it is a vaccine containing killed coxsackie B viruses of at least one serotype selected from the group coxsackie B2 viruses, coxsackie B3 viruses, coxsackie B4 viruses and coxsackie B5 viruses.

16. The vaccine based on coxsackie B viruses as claimed in claim 5, characterized in that it is a vaccine containing weakened and/or killed coxsackie B viruses of the serotypes coxsackie B1 viruses, coxsackie B2 viruses, coxsackie B3 viruses, coxsackie B4 viruses, coxsackie B5 viruses and coxsackie B6 viruses.

17. The vaccine based on coxsackie B viruses as claimed in claim 6, characterized in that
it is a vaccine containing protein and/or nucleotide fragments of at least one coxsackie B virus serotype selected from the group coxsackie B1 viruses, coxsackie B2 viruses, coxsackie B3 viruses, coxsackie B4 viruses, coxsackie B5 viruses and coxsackie B6 viruses.

18. The vaccine based on coxsackie B viruses as claimed in claim 7, characterized in that the vaccine for the prevention of acute lymphoblastic leukemia is suitable for administration in children before completion of the first year of life.

19. The vaccine based on coxsackie B viruses as claimed in claim 8, characterized in that the vaccine for the prevention of acute lymphoblastic leukemia is suitable for administration in children before completion of the sixth month of life.

20. The vaccine based on coxsackie B viruses as claimed in claim 9, wherein the vaccine for the prevention of acute lymphoblastic leukemia is suitable for intramuscular or subcutaneous administration in children before completion of the sixth month of life.
